(19) United States
(12) Patent Application Publication
(10) Pub. No.: US 2012/0115248 A1
(43) Pub. Date: May 10, 2012

(54) METHODS OF DETERMINING THE PRESENCE AND/OR CONCENTRATION OF AN ANALYTE IN A SAMPLE

(76) Inventors: Eric V. Ansyln, Austin, TX (US); Youjun Yang, Austin, TX (US); Balazs Szamosfalvi, Bloomfield Hills, MI (US); Jerry Yee, Beverly Hills, MI (US); Stanley Frinak, Farmington Hills, MI (US)

(21) Appl. No.: 13/342,598

(22) Filed: Jan. 3, 2012

Related U.S. Application Data
(63) Continuation-in-part of application No. PCT/US2010/040543, filed on Jun. 30, 2010.
(60) Provisional application No. 61/222,285, filed on Jul. 1, 2009.

(51) Int. Cl. G01N 33/566; G06F 19/00; G06N 3/02; G01N 30/00

(52) U.S. Cl. 436/501; 422/69; 702/19; 706/15

(57) ABSTRACT

Compositions, methods, and systems for monitoring analyte levels are provided herein. The disclosure provides methods and systems for the real-time monitoring of analytes, such as citrate, calcium, phosphate and magnesium, in a biological fluid in a clinical setting.

### CROSS REFERENCE TO RELATED APPLICATIONS

This application is a continuation-in-part of International Application No. PCT/US2010/040543, filed Jun. 30, 2010, which claims the benefit of U.S. Provisional Application No. 61/222,285, filed Jul. 1, 2009, both of which are incorporated herein by reference.

### BACKGROUND

In an indicator displacement assay, a host/indicator complex exchanges with the targeted analyte to form a host/analyte complex, and thereby releases the indicator. Due to the variation of the environment of the indicator, its signal, usually absorption and/or emission, will be modified.

Sequential injection analysis (SIA) was developed in the late 1980s, and gained wide acceptance within the past two decades. It is a simple yet versatile method for instrumentation based on liquid phase chemistry. Sample processing is automated via computer control, and highly reproducible results are obtained. The entire system can be miniaturized and is suitable for field applications.

Hemodialysis, hemofiltration, or a hybrid of both, namely hemodiafiltration, are renal replacement therapies for patients experiencing kidney failure and can be delivered utilizing a multitude of different equipments. Such treatments remove various toxins from a patient's blood via a concentration gradient, convection, or a combination of both. However, blood may clot when drawn out of a patient's circulation system, especially in the hemofilter. Thus, anticoagulation is usually required. Regional citrate anticoagulation was developed to address this problem because citrate can complex with Ca²⁺ and lower the ionized Ca²⁺, which is an essential cofactor for the initiation of the coagulation cascade. However, in the event of a hemofilter failure to remove citrate and/or in patients with severe liver dysfunction with a failure to metabolize citrate, systemic citrate levels of patients may rise drastically resulting in life threatening ionized hypocalcemia, which in turn may lead to sudden death.

### SUMMARY

The present disclosure relates generally to methods of determining the presence and/or concentration level of an analyte in a sample. More particularly, in some embodiments, the present disclosure relates to methods of measuring the concentration of citrate, ionized calcium, magnesium and/or phosphate in a sample.

In one embodiment, the present disclosure provides a method comprising: providing an analyte; providing an analyte receptor and an indicator, wherein at least a portion of the analyte receptor and the indicator form a receptor/indicator complex; contacting the receptor/indicator complex with the analyte; and allowing the analyte to interact with the receptor/indicator complex so as to generate a detectable signal.

In another embodiment, the present disclosure provides a system comprising: a receptor/indicator complex comprising an analyte receptor and an indicator; and an analyte, wherein the analyte will displace the indicator in the receptor/indicator complex; and wherein the displaced indicator will generate a detectable signal.

The features and advantages of the present disclosure will be readily apparent to those skilled in the art upon a reading of the description of the embodiments that follows.

### DRAWINGS

A more complete understanding of this disclosure may be acquired by referring to the following description taken in combination with the accompanying figures in which:

FIG. 1 is an image depicting a mechanism of analyte sensing via an indicator displacement assay, according to one embodiment.

FIG. 2 is an image depicting a mechanism of analyte sensing via an analyte (Ca²⁺) binding to a receptor/indicator complex (Fura-2), according to one embodiment.

FIGS. 3A and 3B depict the structure of representative citrate receptors, according to one embodiment.

FIG. 4 depicts several representative Ca²⁺ receptors, according to one embodiment.

FIG. 5 depicts several representative Mg²⁺ receptors, according to one embodiment.

FIG. 6 depicts the synthesis scheme of Mg²⁺ receptors 2 and 3 (FIG. 5) from known compounds, according to one embodiment.

FIG. 7 depicts representative Mg²⁺ receptors, according to one embodiment.

FIG. 8 depicts representative phosphate receptors based on H-bpmp, according to one embodiment

FIG. 9 is an image depicting a mechanism of analyte sensing via indicator displacement assay using H-bpmp, according to one embodiment.

FIG. 10 depicts changes of the solution UV-Vis spectra containing a H-bpmp receptor and a pyrocatechol violet indicator upon the addition of a phosphate analyte, according to one embodiment.

FIG. 11 depicts the structure of representative indicators, according to one embodiment.

FIGS. 12A and 12B depict sample calibration curves for citrate (11A) and Ca²⁺(11B), according to one embodiment.

FIG. 13 depicts the working principle of a Flow-Injection-Analysis (“FIA”) instrument, according to one embodiment.

FIG. 14 is a schematic representation of a FIA instrument, according to one embodiment.

FIG. 15 depicts the proposed binding modes of Receptor 2 with alizarin complexone and citrate.

FIG. 16A depicts changes of the solution UV-Vis spectra containing both Receptor 2, and Alizarin Complexone upon addition of citrate, according to one embodiment. Arrows indicate the spectral changes upon increasing citrate concentration.

FIG. 16B depicts the extrapolated calibration curve of citrate concentration by monitoring the absorbance at 540 nm, according to one embodiment.

FIG. 17A depicts changes of the solution UV-Vis spectra containing Fura-2 upon addition of Ca²⁺, according to one embodiment. Arrows indicates the spectral changes upon increasing the Ca²⁺ concentration.

FIG. 17B depicts extrapolated calibration curve of the Ca²⁺ concentration by monitoring the solution absorbance at 375 nm, according to one embodiment.

FIG. 18 is a schematic representation of a SIA analysis of citrate and Ca²⁺ simultaneously, according to one embodiment.

FIG. 19 depicts triple measurements of a sample containing both citrate and Ca²⁺, according to one embodiment.

FIG. 20 depicts calibration curves for citrate and Ca²⁺ using data from SIA system, according to one embodiment.

While the present disclosure is susceptible to various modifications and alternative forms, specific example embodiments have been shown in the figures and are herein described in more detail. It should be understood, however, that the description of specific example embodiments is not intended to limit the disclosure to the particular forms disclosed, but on the contrary, this disclosure is to cover all modifications and equivalents as defined by the appended claims.

### DESCRIPTION

The present disclosure relates generally to methods of determining the presence and/or concentration level of an analyte in a sample. More particularly, in some embodiments, the present disclosure relates to methods of determining the presence and/or concentration level of citrate, ionized calcium, magnesium and/or phosphate in a sample.

In one embodiment, the present disclosure provides a method comprising: providing an analyte; providing an analyte receptor and an indicator, wherein at least a portion of the analyte receptor and the indicator form a receptor/indicator complex; contacting the receptor/indicator complex with the analyte; and allowing the analyte to interact with the receptor/indicator complex so as to generate a detectable signal. In some embodiments, the analyte displaces at least a portion of the indicator in the receptor/indicator complex to form, a receptor/analyte complex. In other embodiments, the analyte may bind to the receptor/indicator complex.

The present disclosure provides methods that may solve many of the clinical problems associated with continuous veno-venous hemofiltration (CVVH) and/or similar procedures by providing methods that enable the monitoring of analyte concentration levels, such as citrate, calcium, magnesium and phosphate, in real time or at regular intervals (such as hourly). For maximum safety, in certain embodiments, the methods may provide a warning of any change in systemic analyte levels so as to prompt the monitoring personnel to review and adjust the treatment settings to ensure the safe continuation of the CVVH or similar procedure. Furthermore, in some embodiments, the methods of the present disclosure may provide information for the fine-tuning of dosages, including calcium plus magnesium dosing, and also monitor the metabolic function of the liver through monitoring the rate of citrate metabolism.

Continuous renal replacement therapy (CRRT) is a form of extracorporeal blood treatment (EBT) that is performed in the intensive care unit (ICU) for patients with acute renal failure (ARF) or end-stage renal disease (ESRD), who are often hemodynamically unstable with multiple co-morbidities. In a specific form of CRRT, continuous veno-venous hemofiltration (CVVH), blood is pumped through a hemofilter and uremic toxin-laden plasma ultrafiltrate is discarded at a rate of 1-10 liters per hour (convective removal of solutes). An equal amount of sterile crystalloid solution (replacement fluid, CRRT fluid) with physiological electrolyte and base concentrations is simultaneously infused into the blood circuit either before the hemofilter (pre-dilution) or after the hemofilter (post-dilution) to avoid volume depletion and hemodynamic collapse.

From a theoretical and physiological point of view, when run continuously for 24 hours per day, CVVH is the closest of all available renal replacement therapy (RRT) modalities today to replicate the function of the native kidneys and the preferred treatment modality for critically ill patients with renal failure. Nevertheless, 90% of RRT in the ICU is performed as intermittent hemodialysis (IHD), sustained low efficiency dialysis (SLED), or sometimes as continuous veno-venous hemo-diafiltration (CVVHDF). Common to all of these latter methods of RRT is that the removal of most solutes is predominantly by the process of diffusion from blood plasma through the membrane of the hemofilter into the dialysis fluid. Diffusion is less efficient in the removal of larger solutes and also provides less predictable small solute movement than convection and therefore, from a theoretical standpoint, CVVH is a superior method of RRT.

The most important reason for the limited use of CVVH in the ICU is that anticoagulation is mandatory to prevent clotting of the extracorporeal circuit in 24-hour treatments. Systemic anticoagulation has an unacceptable rate of major bleeding complications in critically ill patients and cannot be done safely. Similarly, extracorporeal blood treatments including plasmapheresis, plasma adsorption on specialized columns, blood banking procedures, lipid apheresis systems, plasma adsorption-based endotoxin removal, treatment with a bioartificial kidney device that contains live renal tubular cells, or with a liver replacement therapy circuit also require powerful anticoagulation.

Regional citrate anticoagulation (RCA) has emerged as a possible solution to the clinical problem of circuit clotting without inducing any systemic bleeding tendency. Citrate, a trivalent anion, is present in the human plasma as an intermediate of metabolism. This ion chelates ionized calcium in the plasma resulting in a single negative Ca-citrate complex and decreased free ionized calcium levels. Since the coagulation cascade requires free ionized calcium for optimal function, blood clotting in the extracorporeal blood circuit (EBC) can be completely prevented by an infusion of citrate into the arterial (incoming) limb of the EBC. When the blood is passed through the extracorporeal processing unit, the anticoagulant effect can be fully reversed by the local infusion of free ionized calcium into the venous (return) limb of the EBC. Therefore, theoretically, regional citrate anticoagulation can be both very powerful and fully reversible without systemic (intra-patient) bleeding tendencies.

Regional citrate anticoagulation can be performed. Due to the lack of a simple and efficient protocol for the analysis of the critical composition of ultrafiltrate or blood, however, a number of complications associated with the practice of RCA occur. The following complications are well documented: hypernatremia; metabolic alkalosis; metabolic acidosis, hypocalcemia 1 (due to net calcium loss from the patient), hypocalcemia 2 (due to systemic citrate accumulation), rebound hypercalcemia (due to release of calcium from citrate after CVVH is stopped), hypophosphatemia, fluctuating levels of anticoagulation, nursing and physician errors, ionized hypomagnesemia, declining filter performance, trace metal depletion, etc. All these may be solved if real time monitoring of analytes, specifically citrate and ionized calcium is made possible.

Additionally, using a conventional CVVH system, the patient's systemic plasma citrate level can fluctuate in the 0-3 mmol/L range depending on the body metabolism of citrate. Since an accumulation of systemic citrate to 3 mM could result in significant systemic ionized hypocalcemia unless the calcium infusion is increased to proportionally increase the plasma total calcium level, it is necessary to monitor the systemic citrate and total calcium levels.

Laboratory testing of citrate and ionized calcium is not available in the routine clinical ICU setting. Marked changes in citrate and calcium levels can also develop in 2-3 hours during CVVH, too quickly for routine plasma chemistry monitoring every 6 hours to detect such problems in a timely manner before they have adverse clinical sequelae. The effluent fluid contains a wealth of information on the patient's plasma solute composition. This fluid is a clear crystalloid with a small amount of albumin, small peptides, and cytokines also present. The transparency and minimal viscosity of the effluent fluid provide for an ideal environment for an optical- and/or chemical sensor array. However, in current clinical practice, it is discarded without any further analysis.

Furthermore, reduced Mg(II) concentration in blood, known as hypomagnesemia, may lead to weakness, muscle cramps, cardiac arrhythmia, increased irritability of the nervous system with tremors, athetosis, jerking, nystagmus and an extensor plantar reflex. In addition, there may be confusion, disorientation, hallucinations, depression, epileptic fits, hypertension, tachycardia and tetany. However, due to the lack of convenient and reliable clinical monitoring protocol of magnesium, a 2.5:1 molar ratio between total plasma calcium and total plasma magnesium is usually maintained by using a high-Mg commercial replacement fluid. Phosphate losses can also be very large and can quickly lead to severe hypophosphatemia with high daily clearance goals during CVVH unless phosphate is added to the CRRT replacement fluid.

Due to the interaction between citrate and free ionized calcium, the goals of the present disclosure, according to certain embodiments, may be achieved by providing a method to measure the concentration levels of an analyte, such as citrate and/or ionized calcium (e.g., free and/or total ionized calcium) in a sample, such as a bodily fluid. In one embodiment, a receptor and an indicator may be provided in the filter effluent fluid line during CVVH. This allows for the indirect measurement of the analyte level in the patient's systemic blood.

In one embodiment, the methods of the present disclosure may utilize an indicator displacement assay (IDA) for the quantification of an analyte, such as citrate or a different analyte. FIG. 1 contains an image depicting an IDA, according to one embodiment of the present disclosure. As shown in FIG. 1, IDA is a process in which an analyte receptor is initially allowed to form a weakly associated complex with an indicator, such as a chromophore or fluorophore, and reach equilibrium. This equilibrium will be affected when an analyte bearing better structural complimentarity to the receptor than the indicator, is introduced into the system. The receptor/indicator complex will start to diminish allowing the receptor/analyte complex to form. At the same time, the indicator in the cavity of the receptor will be released. Due to the variation of the chemical environment of the indicator, its output signal, usually absorption or emission spectra will be modified. This change may be conveniently used in analysis of the analyte concentration provided necessary parameters describing the related equilibria are known.

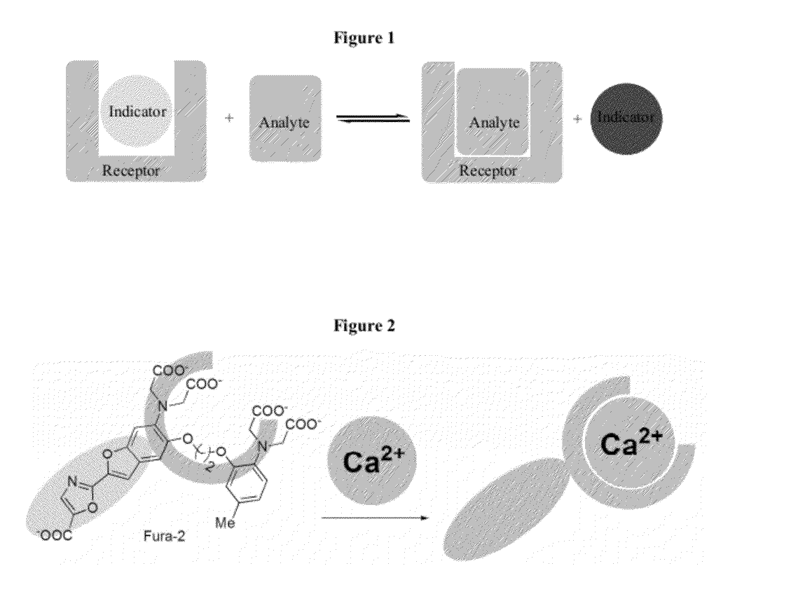

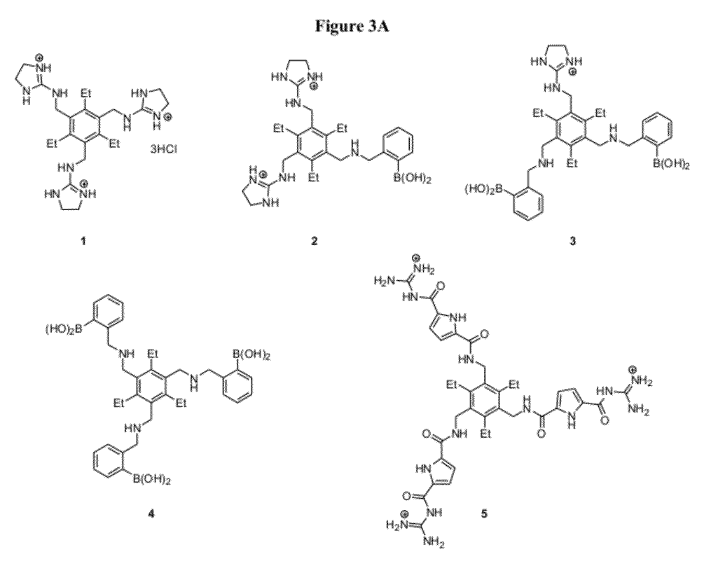

In another embodiment, the present disclosure provides for the detection of an analyte by allowing the analyte to bind to a receptor/indicator complex. After analyte binding, a detectable signal is produced. One example is shown in FIG. 2, which contains an image depicting the binding of ionized calcium to the receptor/indicator complex, Fura-2.

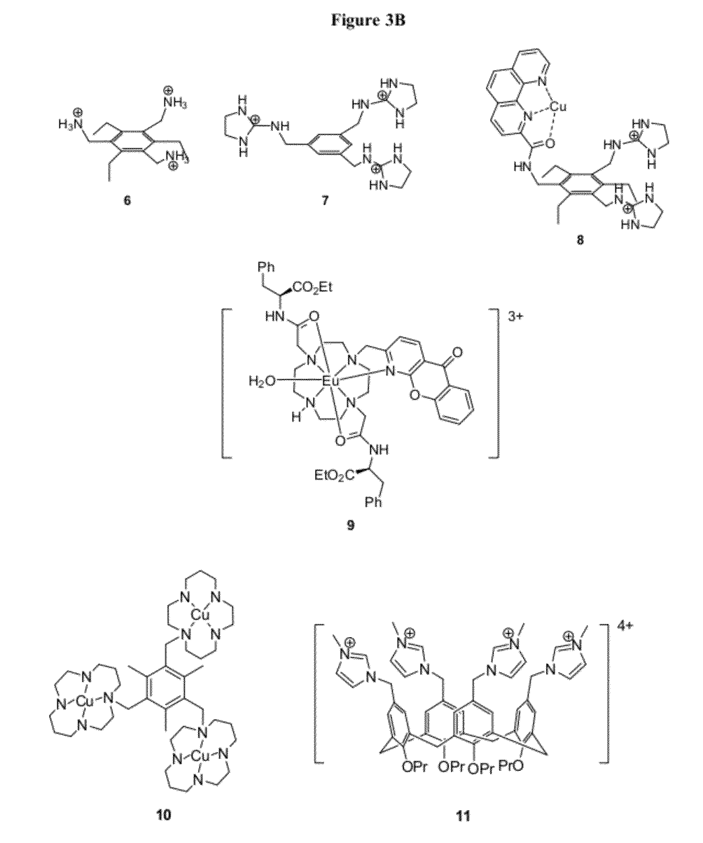

The success of the methods of the present disclosure depend, at least in part, upon the affinity of the receptor or the receptor/indicator complex to bind to the analyte. A variety of different receptors may be used. In certain embodiments, where the analyte is citrate, the receptor is based upon a 2,4,6-triethylbenzene core. However, the receptor can use any scaffold that brings together the functional groups. Various functional groups, including but not limited to guanidinium and phenylboronic acids, are substituted in the 1, 3, and 5 positions. Guanidinium is a favorable functional group because its geometry is conducive for the binding of carboxylates present in citrate and it remains protonated over a wide range pH range. Phenylboronic acid can form robust boronate ester with the α-hydroxy carboxylate moiety of citrate via covalent bonds and represents another favorable functional group for citrate binding. FIGS. 3A and 3B illustrate several representative citrate receptors. Each of these citrate receptors can be easily synthesized by one of skill in the art. Initial trials have shown that Receptor 2 may be a preferred receptor for citrate. The interactions between the citrate receptor and glucose, fructose, or lactate are insignificant enough to be neglected. Other compounds or ions such as bicarbonate, chloride, phosphate and β-hydroxybutyrate are also expected to cause no interferences.

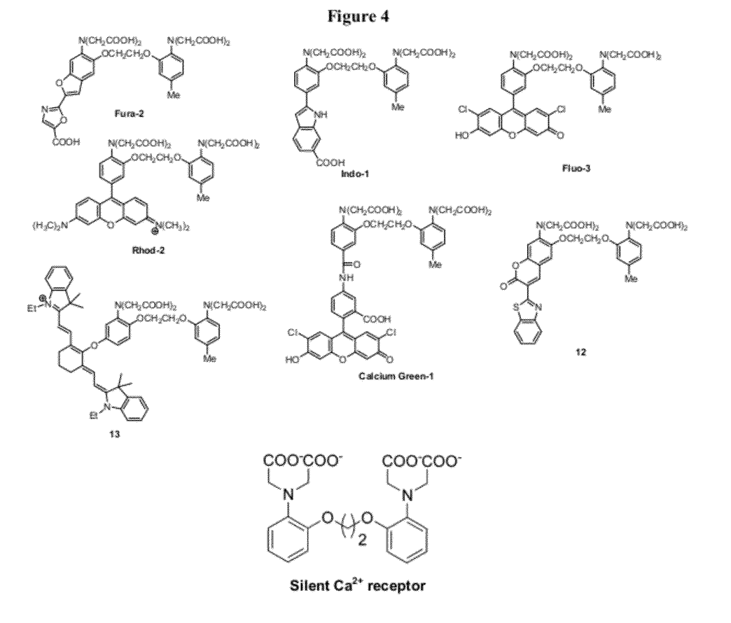

In those embodiments where the analyte is calcium, a variety of Ca²⁺ receptors (only some of which are shown in FIG. 4) may be used and are now commercially available from different vendors. Many of them have the common EDTA-mimicking moiety, which forms a stable complex with Ca²⁺ in solution. When such a moiety is appended to a chromophore or fluorophore, modified spectroscopic properties occur after complexation. In one embodiment, the calcium receptor may be Fura-2, which is commercially available from Invitrogen. Owing to its high complexation constant with Ca²⁺, Fura-2 could extract the Ca²⁺ from the complexes with competing anions, such as citrate³⁻, PO₄³⁻, etc. Additionally, Fura-2 shows high selectivity toward Ca²⁺ over other ions such as Mg²⁺, Na⁺, K⁺, etc. The absorption band of Fura-2 is centered at 273 nm. This allows for the detection of Ca²⁺ to take place essentially free from interferences caused by residual proteins in the dialysis fluid, which produce absorption generally below 330 nm.

In those embodiments where the analyte is magnesium, a variety of Mg²⁺ receptors may be used. As would be recognized by one of skill in the art, most current commercially available Mg²⁺ receptors show higher affinity towards Ca²⁺. Therefore, when choosing an appropriate Mg²⁺ receptor, receptors that show an affinity to Mg²⁺ over Ca²⁺ may be selected.

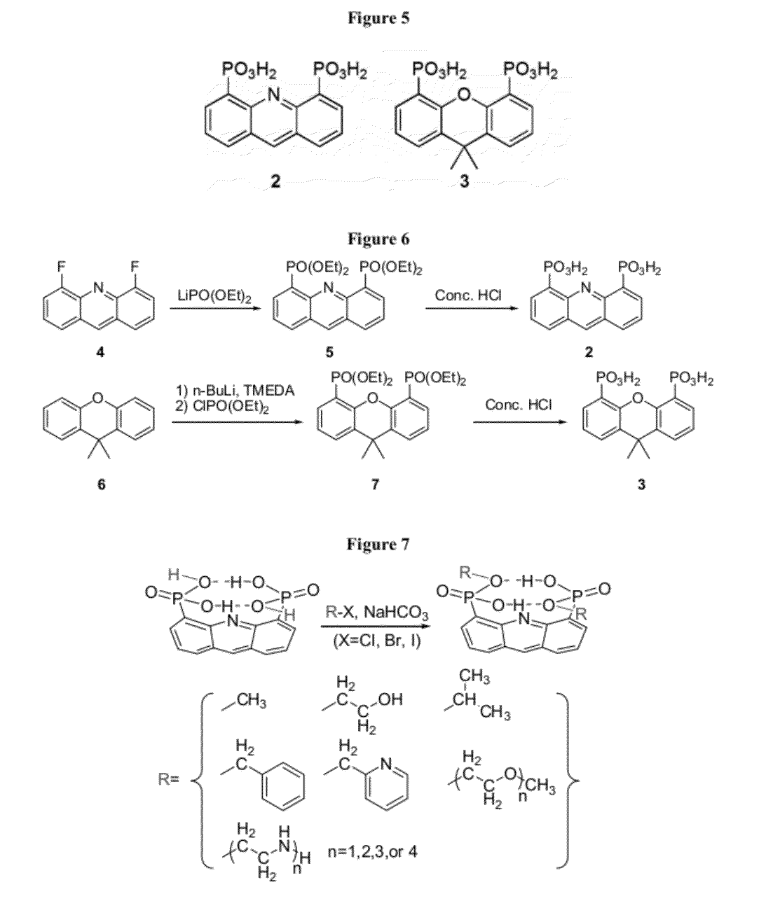

In one embodiment, a suitable Mg²⁺ receptor may include those receptors shown in FIG. 5. Receptors 2 and 3 (from FIG. 5) may be synthesized from the corresponding acridine or xanthene precursors, as shown in FIG. 6. The two fluorine atoms of 4,5-difluoroacridine (4) may be displaced via SN_{AR} mechanism when treated with an appropriate nucleophile. It was reported that negatively charged phophorous species displace fluorine atoms while neutral phosphine does conjugate addition at C-9. Double ortho-lithiation of 9,9-dimethylxanthene (6) is effected by refluxing with n-BuLi and TMEDA in pentane for 10 mins. Quenching with chlorodiehtylphosphate furnishes the ethylphosphonate intermediates. Both 5 and 7 may be easily hydrolyzied by refluxing in concentrated HCl solution to yield the desired receptor 2 and 3, respectively. In addition, the steric and electronic properties of receptors 2 or 3 (from FIG. 5) may be further fine tuned via alkylation of the phosphonate group as shown in FIG. 7.

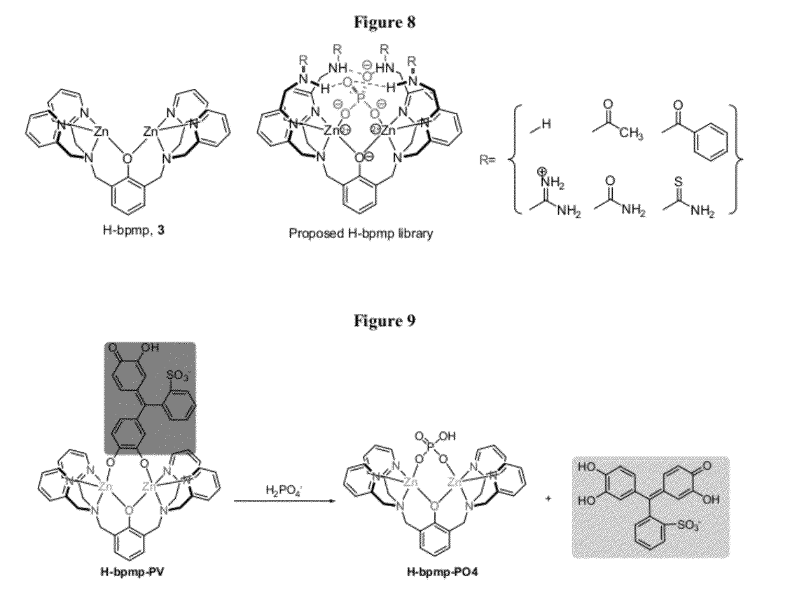

The present disclosure also allows for the testing of phosphate. A number of phosphate receptors with various degrees of selectivity are known in the art. In one embodiment, a suitable phosphate receptor may include those receptors shown in FIG. 8. A preferred embodiment uses H-bpmp as reported in (Han, M. S. et. al. Angew. Chem., Int. Ed. 2002, 41, 3809-3811) because it is reported to display selectivity over common anions, such as chloride, bicarbonates, nitrates, etc. The receptor may be synthesized by following the literature procedures. A sensing mechanism according to one embodiments is shown in FIG. 9. Pyrocatechol violet (PV) can coordinate to the two Zn²⁺ metal centers in the absence of phosphate or other competing anions. Upon addition of phosphate solution, PV will get displaced and changes to its protonation states will cause the solution color to change from light navy to brownish yellow.

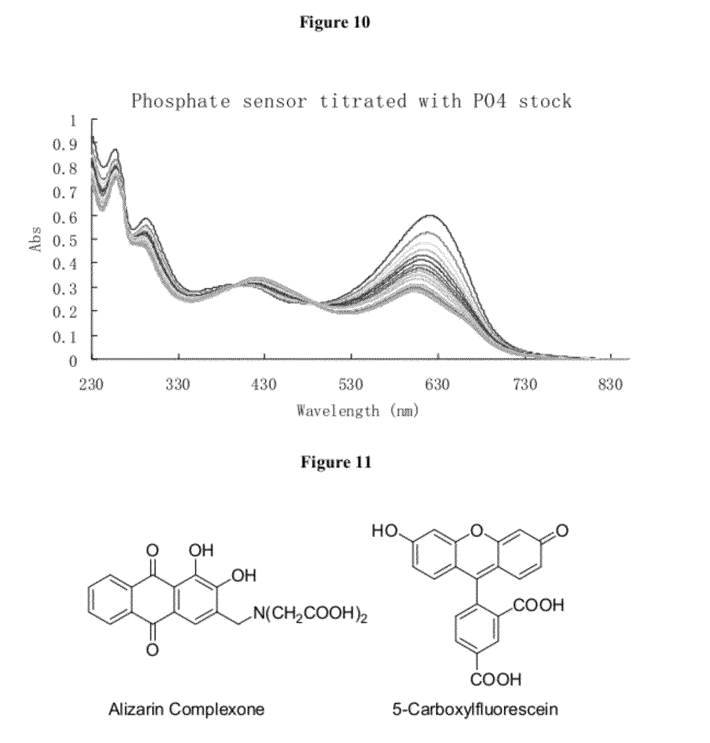

Though phosphate leads to a significant spectral change, as depicted in FIG. 10, initial results have shown that citrate can cause severe interferences to the phosphate sensing due to the fact that citrate displays a higher affinity towards the H-bpmp receptor. In such systems where two interfering variants are to be determined, it would be necessary to introduce the use of pattern recognition. When phosphate sensing is performed, the change to the readout signal is not only determined by the concentration of phosphate but also the concentration of citrate in the solution. The same is also true when citrate sensing is performed, though the influence of phosphate is minimal. A mathematical treatment such as artificial neural network (ANN) processing of the raw data can help extrapolate the actual concentration of both phosphate and citrate. In one embodiment, processing of UV-Vis measurements may be accomplished using Statistica Artificial Neural Network software.

The use of a solvent system comprising 75% MeOH: 25% aqueous buffer (v/v) instead of 100% aqueous buffer solution is found to improve selectivity toward phosphate over citrate. This may lead to higher accuracy in phosphate measurements. Additionally, the stability of the phosphate sensing ensemble solution is also dramatically improved in such solvent system.

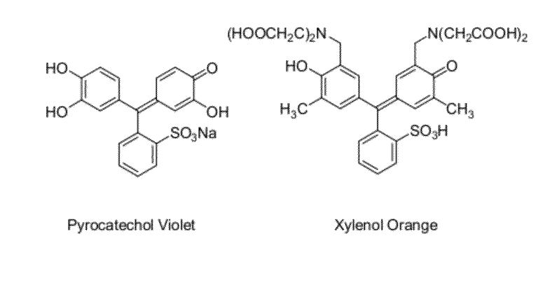

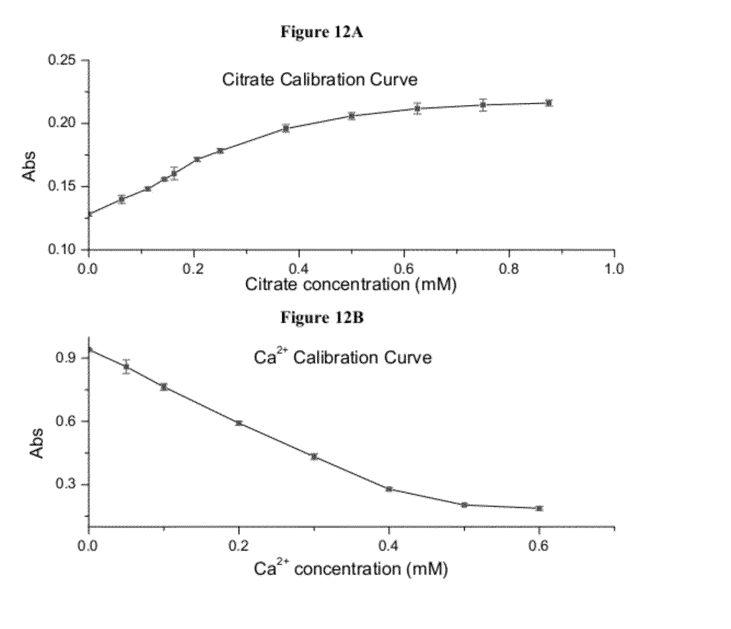

Indicators that are suitable for use in the present disclose include those indicators that are capable of producing a detectable signal when displaced from a receptor/indicator complex by an analyte or those that are capable of producing a detectable signal when an analyte is bound to the receptor/indicator complex. Examples of suitable indicators include, but are not limited to, a chromophore, a fluorophore, alizarin complexone, 5-carboxyfluorescein, pyrocatechol violet, and xylenol orange. FIG. 11 illustrates some representative indicators, which are featured with either a catechol moiety or multiple anionic residues. In one embodiment, alizarin complexone is used as the indicator for analysis of citrate concentrations. Alizarin complexone displays a relatively high binding affinity with Receptor 2 originating from the reversible boronic acid/diol interaction. This interaction between receptor and indicator is strong enough to allow the receptor/indicator complex to form to a great extent thus a large spectral change is attained. This is particularly advantageous in minimizing the errors when performing the citrate concentrate measurement activities. However, the strength of the association is still moderate enough to allow the indicator to be displaced by citrate to essentially completion. A calibration curve may be created by plotting the absorbance of a particular wavelength of light at known concentrations of citrate or another analyte. FIGS. 12A and 12B show representative calibration curves for citrate and calcium. Later, the concentration of an unknown sample may be determined simply by checking the UV-Vis absorption and comparing to the established calibration curve. It is important to account for temperature, however, as temperature affects the equilibrium significantly. Changes of room temperature during the analysis may lead to biased results.

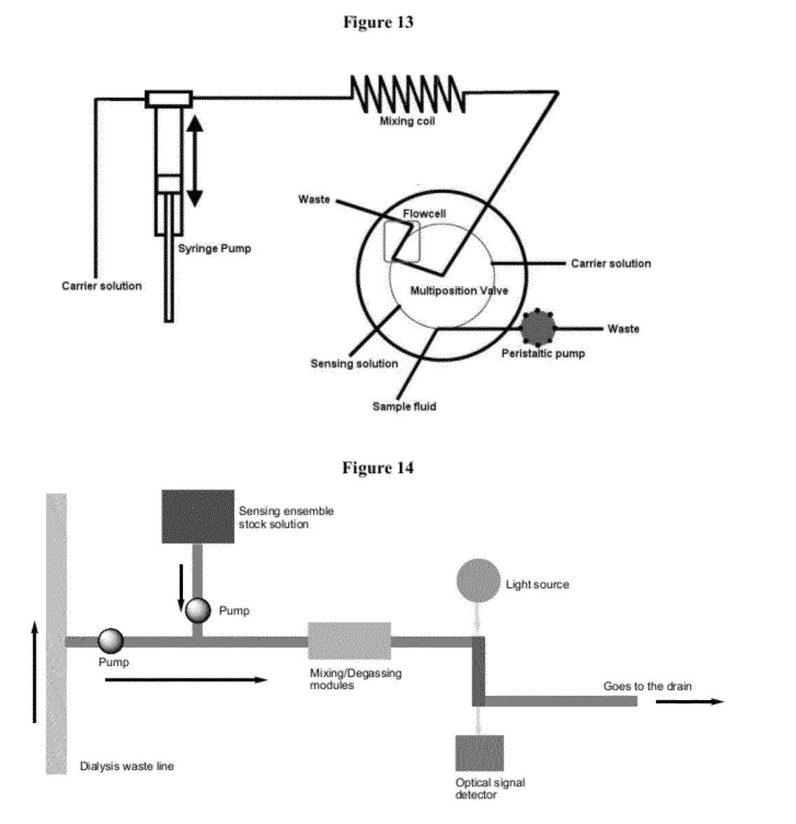

After a detectable signal has been generated, in some embodiments, this signal may be detected through a variety of methods. In one embodiment, the signal may be detected through the use of a spectrometer. In another embodiment, the signal may be detected through the use of a Flow Injection Analysis (FIA) instrument. For example, a Sequential Injection Analysis (SIA) System from Ocean Optics, Inc. may be used. In some embodiments, this method of detection may be particularly advantageous as a general UV/vis spectrophotomer is quite space demanding. The SIA System has dimensions of 5″×6″×6″ and weighs about 8 lbs. It can also automate liquid transferring and mixing with precise control of volumes with the aid of a personal computer. A build-in compact UV-Vis photometer can then acquire the absorption spectra and the obtained data can be simultaneously analyzed. The working principle of this SIA instrument is shown in FIG. 13 and FIG. 18. An aliquot of sensing solution and dialysis fluid is aspirated into the mixing coil before further pushed into the built-in flowcell for optical signal measurement. Such SIA devices allows intermittent measurements to be done in an automatic fashion. The frequency can be as fast as 1 minute or so depending on the programs for a specific application.

In another embodiment, an instrument based on the FIA working principle, for example as depicted in FIG. 14 and FIG. 18, may be used to measure in a continuous, real-time fashion. Such instruments may include a computer, a wireless network, or both to allow, for example, 24 hour online computer monitoring of the ICU dialysis machines using a wireless network. The computer also may be used to automate sample processing. In some embodiments, the entire system may be miniaturized and suitable for field applications. In one embodiment, dialysis fluid to be tested is pumped into a line leading to the Flow-Injection-Analysis (FIA) instrument at a steady speed. An aliquot of sensing ensemble stock solution for a certain analyte, e.g., Ca²⁺, citrate, magnesium, phosphate, etc., is pumped into the line to get mixed with the dialysis fluid and induce an optical signal, which is collected by various detectors, e.g., UV-Vis spectro-photometer, CCD cameras, etc. Though optical signals are generally preferred, other methods may be considered as well if they are advantageous in certain circumstances, including but not limited to near-infrared spectroscopy, Raman spectroscopy, Potentiometry, etc). A degassing module could be of use in case gas bubbles are generated during mixing. Additionally, a sensor (e.g., a hemoglobin sensor) may be included in the blood circuit to determine plasma flow, which may be used to determine the calcium infusion rate.

To facilitate a better understanding of the present disclosure, the following examples of certain aspects of some embodiments are given. In no way should the following examples be read to limit, or define, the entire scope of the disclosure.

### EXAMPLE 1

An aqueous solution containing all the essential components of a typical dialysis fluid, except for citrate, Ca²⁺, Mg²⁺ and CO₂ is prepared. A 100 mM HEPES buffer with pH at 7.40 is prepared from the above stock solution. The citrate sensing ensemble is prepared as following: 1) mixing 75 mL of MeOH and 25 mL HEPES buffer, 2) dissolving the particular amount of citrate Receptor 2 and alizarin complexone to make their concentrations 100 μM and 250 μM respectively.

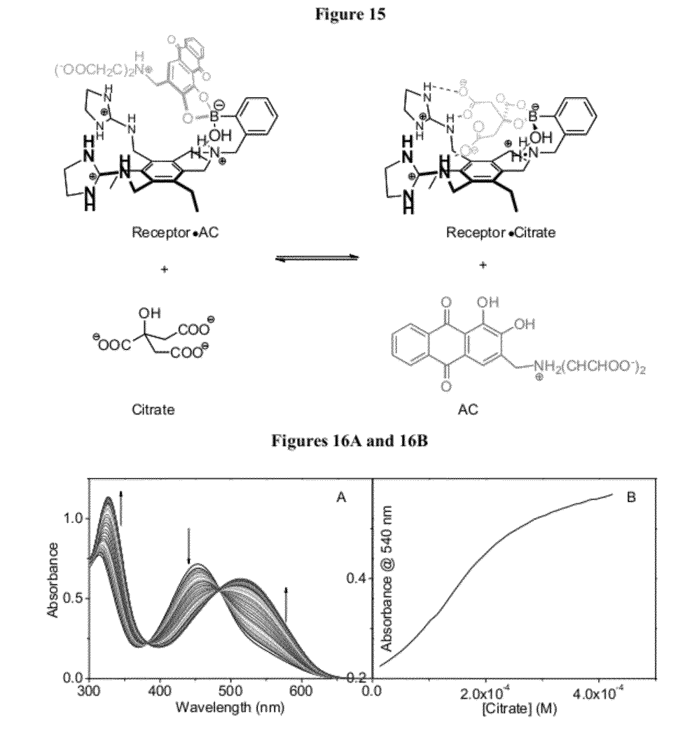

Upon the addition of citrate into the sensing ensemble, alizarin complexone is displaced from the cavity of Receptor 2, yielding to the larger affinity constant between Receptor 2 and citrate. Besides the boronic acid/diol interaction, charge pairing provides an extra driving force for the complexation between the citrate and the Receptor 2 (FIG. 15).

FIG. 16A demonstrates the change in the absorption spectra of Alizarin complexone when in and outside of the receptor cavity. As the citrate concentration increases, absorption maxima of alizarin complexone at 337 nm and 540 nm increase while the maximum at 447 nm decreases. A calibration curve is made by plotting the solution absorbance at 540 nm vs. the corresponding citrate concentration (FIG. 16B).

### EXAMPLE 2

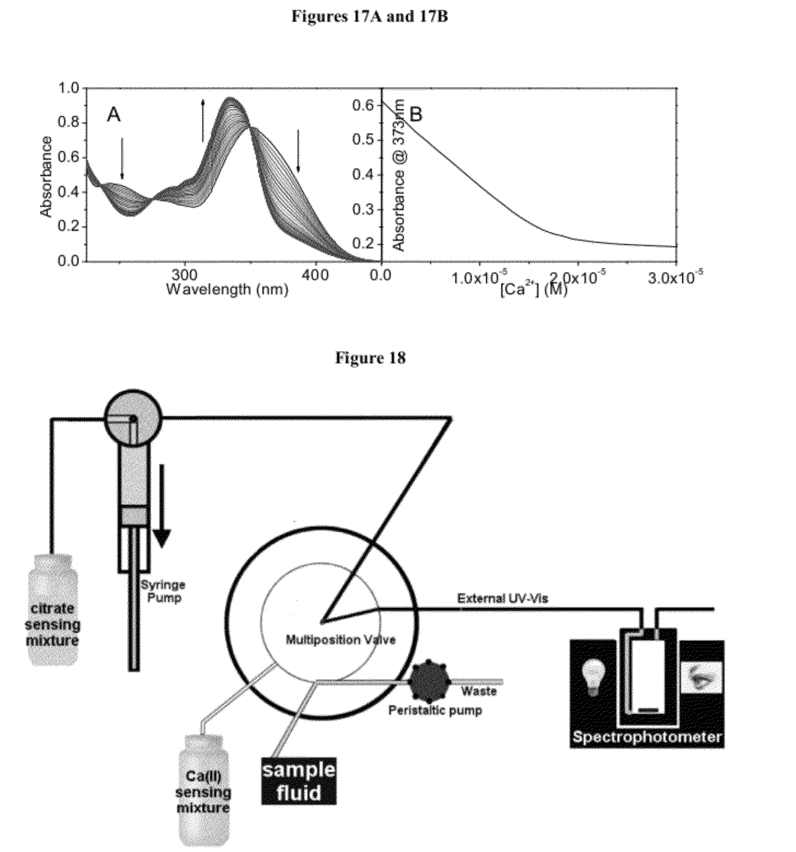

A solution of Fura-2 at 25 μM is prepared using the stock solution mentioned above. An aliquot of sample containing Ca²⁺ is added and changes in the UV-Vis spectrum are observed. As the Ca²⁺ concentration increases, the absorption maxima at 373 nm decreases while the maximum at 330 nm increases (FIG. 17A). A calibration curve is made by plotting the solution absorbance at 373 nm vs. the corresponding Ca²⁺ concentration (FIG. 17B). The Ca²⁺ concentration of an unknown sample may be obtained by its addition into the Fura-2 solution, checking the absorbance at 373 nm and comparing to the calibration curve. Fura-2 displays such a high binding constant with Ca²⁺ that: 1) Mg²⁺, another prevalent divalent cation present in the dialysate fluid, doesn't interfere, 2) citrate, which has a relatively weak binding affinity to Ca²⁺, doesn't displace Fura-2 in Ca²⁺ binding.

### EXAMPLE 3

Eight patient dialysate fluid sample obtained from ICU unit of the Henry Ford Hospital was tested for [Ca²⁺] and [Citrate] using the calibration curves shown in FIGS. 12A and 12B. The Ca²⁺ and citrate concentrations in the dialysate fluid were calculated based on the absorption spectra of the resulting solutions (Table 1).

TABLE 1

| | Sample ID | ICU-1 | ICU-2 | ICU-3 | ICU-4 | ICU-5 | ICU-6 | ICU-7 | ICU-8 |
|---|---|---|---|---|---|---|---|---|---|
| Ca²⁺ | Abs | 0.8895 | 0.9162 | 0.9369 | 0.8986 | 0.8232 | 0.7823 | 0.7511 | 0.7013 |
| data | Stdev | 0.0278 | 0.0281 | 0.0301 | 0.0270 | 0.0278 | 0.0236 | 0.0222 | 0.0249 |
| | VC | 0.031 | 0.031 | 0.032 | 0.030 | 0.034 | 0.030 | 0.030 | 0.035 |
| | Conc (mM) | 0.063 | 0.030 | 0.004 | 0.052 | 0.137 | 0.180 | 0.214 | 0.272 |
| Citrate | Abs | 0.1682 | 0.1682 | 0.1661 | 0.1650 | 0.1850 | 0.1872 | 0.1845 | 0.1720 |
| data | Stdev | 0.0028 | 0.0026 | 0.0022 | 0.0049 | 0.0031 | 0.0032 | 0.0044 | 0.0013 |
| | VC | 0.017 | 0.015 | 0.013 | 0.030 | 0.017 | 0.017 | 0.024 | 0.008 |
| | Conc (mM) | 3.10 | 3.10 | 2.96 | 2.89 | 4.75 | 5.00 | 4.70 | 3.36 |

Notes:
Abs: average absorbance from multiple replicates.
Stdev: standard deviation of the absorbance data from multiple replicates.
VC: coefficient of variation calculated by Stdev/Abs.
Conc: the concentration of the analyte of the interest in the original ICU samples in the unit of millimolar.
ICU samples are diluted with equal amount of 10 mM HEPES buffer at pH 7.4 prior to the Ca²⁺ measurements.
ICU samples are diluted with 3 volumes of 100 mM HEPES buffer at pH 7.4 and 12 volume of MeOH prior to the citrate measurements.

### EXAMPLE 4

Receptor 2 and an IDA was used to construct a prototype instrument and system using sequential injection analysis (SIA) approach.

### Reagents

The citrate Receptor 2 (FIG. 3A) was synthesized with a modified pathway to that published previously. The Ca²⁺ sensor (Fura-2) was purchased from Abd Bioquest. The silent Ca²⁺ receptor (FIG. 4) is from Acros. Alizarin complexone (FIG. 11) was purchased from Aldrich. CaCO₃, NaCl, NaHCO₃, NaOH, HEPES, and trisodium citrate dihydrate were purchased from Fischer Scientific. MeOH was purchased from EMD Biosciences.

### Sample Preparations

A stock solution of NaCl (140 mM) and NaHCO₃ (12 mM) in deionized water (Stock A) was used for the preparation of all aqueous samples. A HEPES buffer (100 mM, pH ¼ 7.4) was prepared by dissolving HEPES in Stock A followed by pH adjustment with a NaOH solution (6 M). The citrate sensing ensemble solution was prepared by mixing 1 (28.5 mg), 3 (8.8 mg), HEPES stock (50 mL), and MeOH (150 mL). The Fura-2 stock was prepared by dissolving Fura-2 (1 mg) in HEPES stock (6 mL) and MeOH (18 mL). The Fura-2 stock for SIA was prepared by dissolving Fura-2 (1 mg) and 2 (0.57 mg) in the HEPES stock (1.2 mL). The Ca²⁺ and citrate standard solutions were prepared by mixing Ca²⁺ stock solution (20 mMin the stock A) and trisodium citrate dihydrate stock solution (80 mM in HEPES buffer) in the above HEPES buffer stock solution.

### Instruments

Spectroscopic studies were performed on a Beckman Coulter DU 800 UV-Vis spectrophotometer. The prototype SIA system was assembled with a MicroSIA from FIAlabs, Inc., powered by FIALab for windows 5.0, a modified commercial flow cell (Catalog number: 583.65.65/Q/10/Z/15) from Starna Inc., and a miniaturized CHEMUSB4 UV-VIS Spectrometer; from Ocean Optics, Inc., powered by logger pro 3 from Vernier Software and Technology. A 3 mm diameter hole was drilled on one side of the flow cell and a micro-stirbar (2×5 mm) was placed in the flow cell and then sealed with a customized Teflon plug.

### Evaluation of the Citrate and Ca²⁺ Sensing Chemistry

We synthesized a series of citrate receptors that were reported in the literature and found that the Receptor 2 from our own group displayed superior affinity toward citrate in the solvent system of 25% HEPES (pH ¼ 7.4, 100 mM) in MeOH (v/v). To establish a satisfactory indicator displacement assay (IDA) for citrate measurements, a number of commercially available indicators were tested: alizarin complexone, 5-carboxyfluorescein, pyrocatechol violet, and xylenol orange (FIG. 11). Alizarin complexone (AC) displayed a relatively high binding affinity with Receptor 2 originating from the reversible boronic acid/diol interaction, and a large spectral change was obtained. However, the interaction was of moderate enough affinity to allow the indicator to be displaced efficiently by citrate (FIG. 15). Addition of citrate standard solutions into the sensing ensemble of 1 and AC caused the absorption band at 485 nm to decrease while bands at 335 nm and 535 nm increased. A calibration curve was made by plotting the solution absorbance at 540 nm against the corresponding citrate concentration (FIG. 16).

We have previously reported an analogous two-component sensing ensemble for the simultaneous measurements of citrate and Ca²⁺ in various flavored vodkas using artificial neural networks (ANN), taking advantage of the cross-reactivity of xylenol orange to both the citrate receptor used therein and Ca²⁺. The same strategy could be applied to dialysis because AC displays such cross-reactivity as well. However, a method that does not require a sophisticated mathematical model would be desirable due to simplicity.

We therefore introduced an independent Ca²⁺ receptor into the citrate sensing ensemble, Fura-2. Fura-2 displays a much higher affinity (K_{d} ¼ 0.1 mM)₈ toward Ca²⁺ over citrate (K_{d} ¼ 0.7 mM). Thus, citrate may be measured without any interference from Ca²⁺ if enough Fura-2 is present for Ca²⁺ chelation. Fura-2 was developed by integrating a high affinity Ca²⁺ ligand (colored in gray) to an oxazole-benzofuran chromophore. Binding of the Fura-2 to Ca²⁺ induced changes to the ionization state of the chromophore and hence the UV-Vis absorption spectrum (FIG. 2). With increasing Ca²⁺, the absorption band at 370 nm decreases and that at 325 increases. A calibration curve was made by plotting the solution absorbance at 370 nm against the corresponding Ca²⁺ concentration (FIG. 17).

Both the Fura-2 and Fura-2/Ca²⁺ complex do not display any optical absorbance above 450 nm, and therefore citrate quantification using an absorbance at 535 nm has no interference. Further, 385 nm is an isosbestic point in the citrate analysis, while Ca²⁺ induces a significant spectral change at this wavelength. Therefore, the Ca²⁺ concentration was monitored using the absorbance at 385 nm even if it is not the wavelength yielding the maximum absorbance change for Fura-2.

It is noteworthy to point out that the presence of phosphate, Mg²⁺, or CO2 in the dialysate does not result in noticeable spectral changes of the citrate sensing ensemble and Fura-2. To avoid errors to the measured citrate value, the upper limit of Ca²⁺ in the sample should be calculated based on the concentration of Fura-2 using eqn (1) assuming a stoichiometric complexation between Fura-2 and Ca²⁺.

$${[ {Ca}^{2 +} ]}_{upper} = \frac{{volume}_{Fura \text{-} 2} \times [ Fura \text{-} 2 ]}{{volume}_{sample}} \qquad ( 1 )$$

The silent Ca²⁺ receptor (FIG. 4) is essentially Fura-2 without the signaling chromophore, and is expected to display similar binding properties towards Ca²⁺. The use of silent Ca²⁺ receptor along with Fura-2 may be preferred when a large amount of Fura-2 is necessary to chelate all the Ca²⁺ present, and when the cost of Fura-2 becomes a concern. The upper limit of Ca²⁺ in this case should be determined using eqn (2).

$${[ {Ca}^{2 +} ]}_{upper} = \frac{{volume}_{Fura \text{-} 2} \times ( [ Fura \text{-} 2 ] + [ receptor 2 ] )}{{volume}_{sample}} \qquad ( 2 )$$

### Automated Quantification of Citrate and Ca²⁺ Using SIA System

Implementation of the developed citrate and Ca²⁺ sensing technology with an automated instrument is particularly important for its adoption by potential end users. Thus, the following system based on the SIA was devised. A high precision syringe pump, a multiposition valve and a miniaturized UV-Vis spectrophotometer equipped with a flow cell were used (FIG. 18). The syringe pump connects the citrate sensing stock and the multiposition valve, which connects to the Ca²⁺ sensing stock, dialysate sampling loop, and UV-Vis spectrophotometer through the various ports available. The syringe pump delivers an accurate amount of citrate sensing solution, Ca²⁺ sensing solution, and dialysate sample to be tested into the flow cell where the reactions occur. The UV-Vis spectrophotometer monitors the absorbance at the wavelength of 385 nm and 535 nm constantly for Ca²⁺ and citrate quantification, respectively. The codes to drive the system are shown in the ESI.

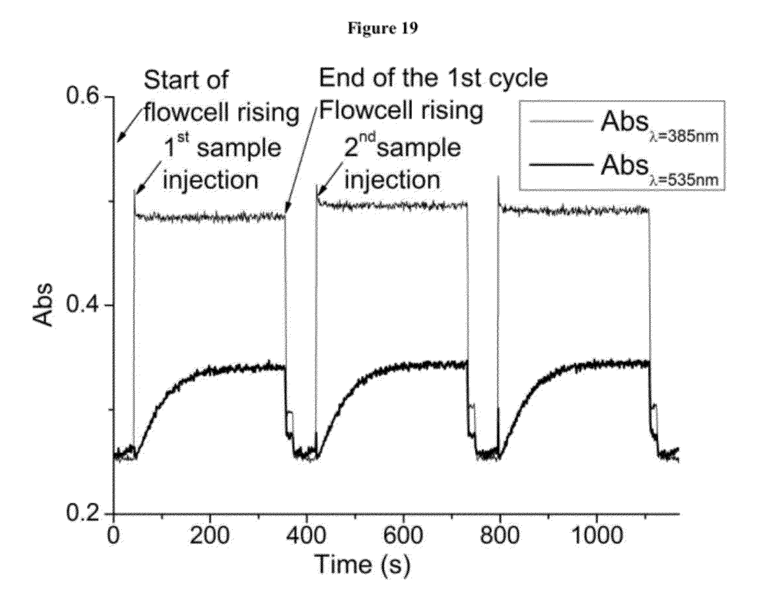

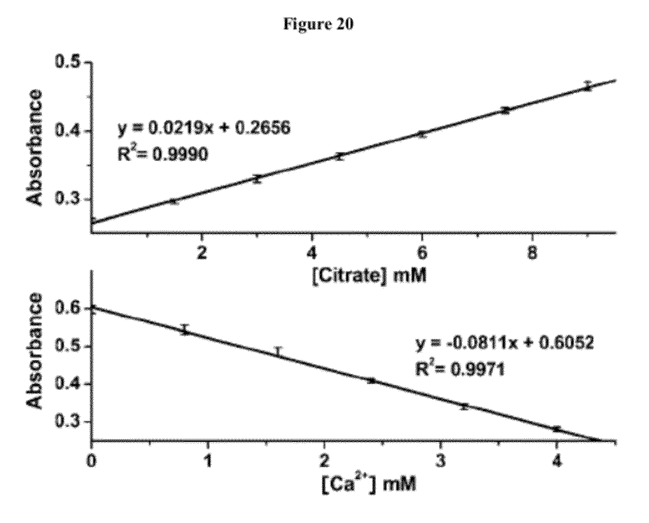

A set of representative data from the SIA system is shown in the FIG. 19. At t ¼ 0 s, flow cell was rinsed three times with citrate sensing ensemble solution. The syringe pump delivers designated volumes of various liquid components, which were consequently injected into the flow cell at t ¼ 40 s. The mixture was stirred by the micro-stirbar in the flow cell and a homogenous solution resulted. The complexation between Fura-2 and Ca²⁺ is complete within ca. 15 s, while it takes around 300 s before the citrate IDA reaches equilibrium, as indicated by the absorbance change at 385 nm and 535 nm respectively. Averaged absorbance values at both wavelengths were recorded prior to the rinsing of the flow cell for further tests. A coefficient of variation of less than 2.5% was obtained. A series of citrate and Ca²⁺ standard solutions are used to establish calibration curves (FIG. 20).

Dialysate samples were obtained from a patient hemodialysis system (Henry Ford Hospital in Detroit, Mich.) and tested for Ca²⁺ and citrate using the SIA method to give [Ca²⁺]_{SIA} and [Cit]_{SIA} (Table 2). Good correlation between [Ca²⁺]_{SIA} and the values measured via atomic absorption methods ([Ca²⁺]_{AA}) was found. A less than 15% error {([Ca²⁺]_{SIA}−[Ca²⁺]_{AA})/[Ca²⁺]_{AA}} was consistently observed. The same samples was also submitted to the local analytical laboratory of Henry Ford Hospital for citrate quantification ([Citrate]_{EA}) using a commercially available enzymatic assay from R-Biopharm. Significant discrepancies were noticed for samples #1, #2, and #6 between [Cit]_{SIA} and [Cit]_{EA}:, while others displayed an error smaller than 15%. To clearly validate the [Citrate]_{SIA} method the same samples were sent to an outside laboratory for analysis via nuclear magnet resonance (NMR) assay for citrate concentrations ([Citrate]_{NMR}). Better agreement was found between [Citrate]_{SIA} and [Citrate]_{NMR} for samples #2 and #6 confirming that the SIA method provides reliable readings for citrate.

TABLE 2
The citrate and Ca²⁺ concentrations measured via different methods

| # | [Ca²⁺]_{SIA} | [Ca²⁺]_{AA} | [Citrate]_{SIA} | [Citrate]_{EA} | [Citrate]_{NMR} |
|---|---|---|---|---|---|
| 1 | 2.30 | 2.37 | 0.66 | 0.18 | n/a^{a} |
| 2 | 0.97 | 1.14 | 1.70 | 0.62 | 1.55 |
| 3 | 0.10 | 0.20 | 0.56 | 0.60 | n/a^{a} |
| 4 | 2.12 | 2.01 | 3.06 | 2.87 | 2.63 |
| 5 | 2.33 | 2.34 | 0.78 | 0.56 | 0.58 |
| 6 | 1.21 | 1.41 | 2.85 | 3.94 | 3.06 |
| 7 | 0.15 | 0.24 | 0.74 | 0.64 | 0.73 |
| 8 | 2.22 | 2.40 | 5.85 | 6.57 | 5.99 |
| 9 | 1.94 | 1.81 | 2.74 | 2.51 | n/a^{a} |
| 10 | 0.48 | 0.75 | 0.93 | 0.92 | 0.92 |

^{a}Note:
[Citrate]_{NMR} was not available for samples #1, #3 and #9 because the solids obtained by lyophilizing the dialysate samples were only partially soluble in Deuterium Oxide for NMR analysis.

A simultaneous citrate and Ca²⁺ quantification method via an IDA and Fura-2 was developed. The use of sophisticated mathematical software to aid in data analysis was avoided in the current method due to the orthogonality between the citrate and Ca²⁺ sensing chemistry. We also developed an automated SIA system and instrumentation, which can be coupled to a hemodialyzer for online monitoring of citrate and Ca²⁺ levels. Data obtained from our SIA system agree well with other methods.

Therefore, the present disclosure is well adapted to attain the ends and advantages mentioned as well as those that are inherent therein. While numerous changes may be made by those skilled in the art, such changes are encompassed within the spirit of this disclosure as illustrated, in part, by the appended claims.

What is claimed is:

1. A method comprising:
providing an analyte;
providing an analyte receptor and an indicator, wherein at least a portion of the analyte receptor and the indicator form a receptor/indicator complex;
contacting the receptor/indicator complex with the analyte; and
allowing the analyte to interact with the receptor/indicator complex so as to generate a detectable signal.

2. The method of claim 1 wherein the analyte is present in a biological fluid.

3. The method of claim 2 further comprising monitoring a concentration level of the analyte present in the biological fluid.

4. The method of claim 2 wherein the biological fluid comprises an extracorporeal blood circuit effluent fluid produced on a hemofilter or a dialyzer device with hemofiltration or dialysis or any combination of these two processes.

5. The method of claim 1 further comprising detecting the detectable signal by using a Flow Injection Analysis instrument.

6. The method of claim 1 further comprising correlating the detectable signal with a calibration curve to determine a concentration of the analyte.

7. The method of claim 1 wherein the analyte is selected from the group consisting of:
ionized calcium, citrate, phosphate, magnesium, and combinations thereof.

8. The method of claim 1 wherein allowing the analyte to interact with the receptor/indicator complex comprises allowing the analyte to displace at least a portion of the indicator in the receptor/indicator complex to form a receptor/analyte complex.

9. The method of claim 1 wherein allowing the analyte to interact with the receptor/indicator complex comprises allowing the analyte to bind to the receptor/indicator complex.

10. The method of claim 1 wherein the indicator comprises at least one indicator selected from the group consisting of: a chromophore, a fluorophore, alizarin complexone, 5-carboxyfluorescein, pyrocatechol violet, xylenol orange, and combinations thereof.

11. The method of claim 1 wherein the analyte receptor is Fura-2 or

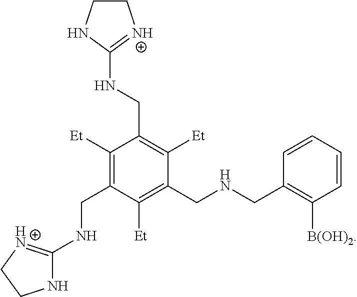

12. The method of claim 1 further comprising using a mathematical treatment to extrapolate the concentration of the analyte.

13. The method of claim 12 wherein the mathematical treatment comprises an artificial neural network (ANN).

14. A system comprising:
a receptor/indicator complex comprising an analyte receptor and an indicator; and
an analyte, wherein the analyte will displace the indicator in the receptor/indicator complex; and wherein the displaced indicator will generate a detectable signal.

15. The system of claim 14 wherein the analyte is present in a biological fluid.

16. The system of claim 14 wherein the analyte is present in a biological fluid, and wherein the biological fluid is an extracorporeal blood circuit effluent fluid produced on a hemofilter or dialyzer device with hemofiltration or dialysis or any combination of these two processes.

17. The system of claim 14 further comprising a Flow Injection Analysis instrument.

18. The system of claim 14 wherein the analyte is selected from the group consisting of:
ionized calcium, citrate, phosphate, magnesium, and combinations thereof.

19. The system of claim 14 wherein the indicator comprises at least one indicator selected from the group consisting of: a chromophore, a fluorophore, alizarin complexone, 5-carboxyfluorescein, pyrocatechol violet, xylenol orange, and combinations thereof.

20. The system of claim 14 wherein the analyte receptor is Fura-2 or

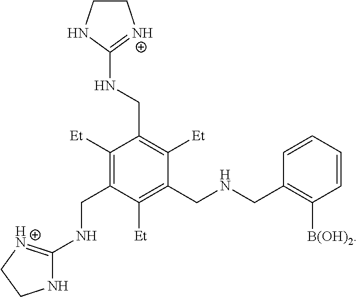

21. The system of claim 14 further comprising one or more of a computer, a wireless network, a hemodialyzer, a plasma flow sensor, a detector, a UV-Vis spectro-photometer, a flow cell, a syringe pump, a multiposition valve, and a peristaltic pump.
